(19) United States
(12) Patent Application Publication
(10) Pub. No.: US 2016/0320308 A1
(43) Pub. Date: Nov. 3, 2016

(54) Tobacco Smoke Detector

(71) Applicant: Jonathan Liss, Columbus, GA (US)

(72) Inventor: Jonathan Liss, Columbus, GA (US)

(21) Appl. No.: 14/698,790

(22) Filed: Apr. 28, 2015

(51) Int. Cl. G01N 21/78; C12Q 1/25; G01N 31/22

(52) U.S. Cl. CPC G01N 21/783; G01N 31/223; C12Q 1/25; G01N 2021/7796

(57) ABSTRACT

A disposable, self-contained tobacco smoke detector, comprising a sensor incorporated with a reagent capable of undergoing one or more chemical reactions by interacting with one or more compounds unique to tobacco smoke. The smoking detector further comprises an indicator coupled to the sensor for visually indicating the occurrence of at least one chemical reaction by producing a detectable change in color, indicating exposure to tobacco smoke, thereby providing visual evidence of smoking.

### FIELD OF THE INVENTION

The present invention generally relates to smoke detectors, and more particularly relates to a disposable, self-contained tobacco smoke detector for visually indicating the presence of tobacco smoke from cigars, cigarettes and the like, in a confined area.

### BACKGROUND OF THE INVENTION

Harmful effects of cigarette smoking are well known as smoking poses a significant threat to the health of smokers and to the health of people around them. Second hand smoke or passive smoking prevails as a major source of morbidity and mortality. Numerous strategies are being implemented to reduce public exposure to environmental tobacco smoke (ETS). These strategies include banning of smoking in public places, such as schools, hospitals and government offices, restaurants, parks etc.

In addition, the rental industry, which includes hotel, motel and automobile rental agencies are also implementing smoke free policies in order to prevent smoking related damage to property, wherein the foul odor of tobacco products renders the property less enticing to prospective clients. Despite the efforts of property management to restrict smoking, some tenants or occupants will violate no-smoking policies. This can lead to costly clean-up that includes de-odorizing; removal of nicotine stains; replacement of accessories like curtains, bed linens, pillows, carpet, seat covers, etc., that might emit the foul odor of tobacco smoke. In addition, the lingering effect of tobacco smoke in a closed environment results in an offensive odor that may trigger nausea, asthmatic exacerbations, allergies, migraines etc. Further, the fact that the room or car cannot be rented until a thorough clean-up or repair has been completed, results in a loss of future revenue.

Enforcing the policy of “No Smoking”, is often hindered with numerous challenges. Current systems or methods for ensuring smoke free premises especially at hotels or motels include manual inspection of rooms by staff members for the presence of evidence such as tobacco ash or cigarette butts or tobacco smoke, periodically or during check out. Disputes arise when the management attempts to recover costs by charging penalties for violating the rules of no smoking, as guests often try to deny or cover-up smoking. Moreover, appropriate legal action by management requires substantial proof or evidence indicating smoking by the occupant, tenant or lessee. Installation of smoke detectors or alarm systems can detect the presence of smoke. However, standard smoke detectors are primarily used to alert everyone in the vicinity that a fire risk is present. This sound is intended to disturb as many people as possible and trigger a potential evacuation along with a search for the source of smoke. The sounding of these kinds of alarms can make a vacation stay less enjoyable and therefore the property less appealing for future rentals. In addition, once the smoke has dissipated the alarm will become silent and a record of the event may not be recorded and the origin of the alarm may never be known, such as which room the alarm erupted. Additionally, standard room smoke detectors are easily disabled and there is universal knowledge that if the smoke substantially avoids the device, an alarm will not sound. Thus, there is very little mystery surrounding a standard smoke detector to offer any additional psychological barriers to would-be violators of no-smoking rules.

Smoke detectors and alarm systems of various designs are common and well known. However, there is a lack of disposable, affordable, silent, and self-contained smoking detectors capable of visually indicating the presence of tobacco smoke or compounds specific to tobacco smoke but not environmental gases or other smoke, thus avoiding false positives. The visual indication means that tobacco smoke has been present and will offer proof or evidence of smoking within a confined area without disturbing the renter, lessee or those people in surrounding units.

Therefore, there still exists a need for a portable, affordable, silent, disposable, and easily replaced smoke detector capable of providing visual evidence of smoking, by detecting the presence of tobacco smoke or tobacco smoke related components within a confined area such as a room or vehicle.

### SUMMARY OF THE INVENTION

The present invention relates to a disposable, self-contained tobacco smoke detector, comprising: a sensor incorporated with a reagent capable of undergoing at least one chemical reaction by interacting with one or more compounds unique to tobacco smoke; and an indicator coupled to the sensor for visually indicating that at least one tobacco related chemical reaction has occurred, by producing a detectable change in color, thereby indicating an exposure of the smoke detector to tobacco smoke or related compounds.

In an embodiment, the tobacco smoke detector of the present invention comprises a sensor integrated with an indicator comprising a reagent capable of interacting with one or more compounds unique to tobacco smoke and producing a visible color change. The chemical reaction will be otherwise inert to avoid any environmental risk of exposure, fire, or harm of any meaningful sort. The detector allows efficient monitoring of confined areas for smoking activities by detecting the presence of tobacco smoke or smoke related compounds. It will also function effectively as a visual deterrent to would-be smokers that they are likely to be provably exposed and therefore reduce the total number of violators by its mere presence. In addition, the tobacco smoke detector provides visual evidence for the act of smoking within a confined area, where smoking is prohibited.

### BRIEF DESCRIPTION OF DRAWINGS

FIG. 1A illustrates a perspective view of the tobacco smoke detector, according to an embodiment of the present invention.

FIG. 1B illustrates a sectional view of the tobacco smoke detector of FIG. 1A.

### DETAILED DESCRIPTION OF THE INVENTION

The following detailed description of the preferred embodiments presents a description of certain specific embodiments to assist in understanding the claims. However, the present invention is intended to cover alternatives, modifications and equivalents, which may be included within the spirit and scope of the invention as defined by the appended claims.

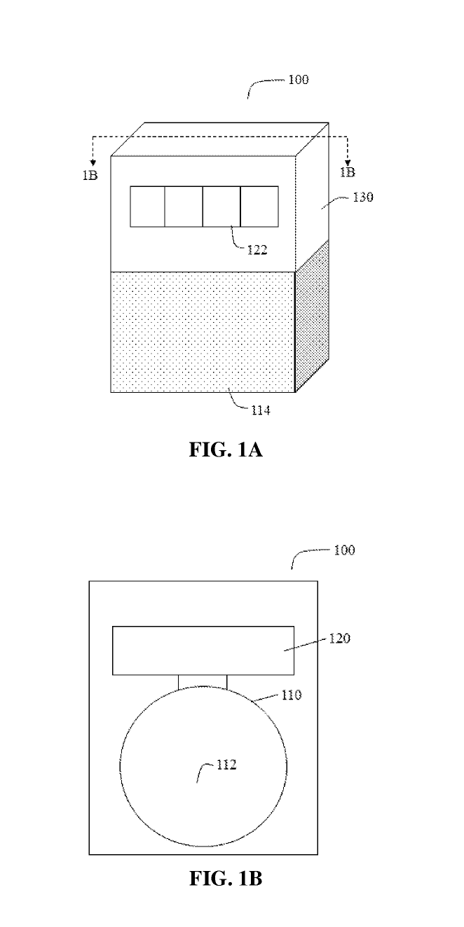

Referring to FIGS. 1A and 1B, illustrating a perspective view and sectional view respectively of the tobacco smoke detector according to an embodiment. The tobacco smoke detector 100 comprises a sensor 110, incorporated with a reagent 112 capable of undergoing at least one chemical reaction by interacting with one or more compounds unique to tobacco smoke. The smoke detector 100 further comprises an indicator 120 coupled to the sensor 110 for visually indicating that at least one chemical reaction specific to tobacco smoke has occurred by producing a detectable change in color. In an embodiment, the indicator 120 is integrated within the sensor 110. The tobacco smoke detector 100 comprises a self-contained housing 130 encasing the sensor 110 coupled with the indicator 120.

The reagent 112 may comprise a biological or chemical component including one or more enzymes, proteins or chemical substances, capable of reacting with one or more compounds unique to tobacco smoke for producing a detectable color change indicating at least one chemical reaction between the sensor substance and the tobacco smoke. In an embodiment, the reagent 112 may comprise one or more enzymes which catalyze one or more chemical reactions in the presence of components unique to tobacco smoke, in order to yield a colored product, which is visible through the indicator window 122. Thus, a color change as indicated by the indicator 120 provides visual evidence of tobacco smoking.

In an embodiment, the indicator 120 is integrated with the sensor unit 110, adapted to react with one or more compounds unique to tobacco smoke and produce a noticeable color change, indicating exposure to tobacco smoke. In another embodiment, the reagent 112 undergoes a series of chemical reactions to produce a colored product, upon interacting with tobacco smoke or smoke related compounds.

The housing 130 is a self-contained unit which can be placed in confined areas for detecting the act of smoking and the indicator 120 provides visual evidence for the presence of tobacco smoke in the confined area. For example, the housing 130 may comprise a configuration resembling a pack of playing cards, the housing 130 may comprise a plurality of openings or apertures 114 allowing the smoke molecules to pass through and reach the sensor 110. The housing 130 may comprise a tamper proof design, preventing tampering by smokers or miscreants. At least one face of the housing 130 may comprise an adhesive layer for affixing the tobacco smoke detector onto flat surfaces such as a wall or a cupboard. Further, the housing 130 may comprise a transparent window 122 providing a clear visibility to the indicator 120. In an embodiment, the indicator 120 or the transparent window 122 is graduated with markings representing the intensity of the tobacco smoke detected.

The portable and compact configuration of the tobacco smoke detector helps in wide range of usage. The tobacco smoke detector of the present invention can be placed in confined areas such as rooms of hotels/motels, cars or other vehicles to be rented, where smoking is restricted or prohibited. It will also function effectively as a visual deterrent to would-be smokers that they are likely to be provably exposed and therefore reduce the total number of violators by its mere presence. The visual indicator provides an undisputed evidence of smoking by occupant or guest. For example, when renting a car or a hotel room, an agreement can be made noting that a tobacco smoke detector is present and that it has been confirmed to be in an unreactive state. When returning the car or after check-out from the hotel, the staff may check for the reactive status of the detector and if a spent device is found, then appropriate cleaning charges or penalty charges can be added to the bill.

In an exemplary embodiment, the tobacco smoke detector comprises a self-contained housing configuration resembling a pack of playing cards, which can be installed or placed in confined locations where tobacco smoking is to be monitored. The housing may comprise a biological or chemical component such as an enzyme which produces a detectable color change upon reacting with one or more compounds unique to tobacco smoke. The detectable color change as indicated by the tobacco smoke detector can be used as evidence of smoking within that confined location.

The tobacco smoke detector can be used in places like rooms of houses, hotels, motels, cars, recreational vehicles in order to detect smoking. The tobacco smoke detector is capable of detecting tobacco smoke or smoke related compounds from cigarettes, cigars, smoking pipes, and the like.

The foregoing description and drawings comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions and the associated drawings. Although specific terms may be employed herein, they are used in a generic and descriptive sense only and not for purposes of limitation. Accordingly, the present invention is not limited to the specific embodiments illustrated herein, but is limited only by the following claims.

1. A disposable, self-contained, tobacco smoke detector, comprising:
a sensor incorporated with a reagent capable of undergoing at least one chemical reaction by interacting with one or more compounds unique to tobacco smoke; and
an indicator coupled to the sensor for visually indicating the occurrence of the at least one chemical reaction by producing a detectable change in color so as to form a detectable color change; and
wherein when the reagent interacts with the one or more compounds unique to the tobacco smoke, the at least one chemical reaction formed thereby has the detectable color change made thereto, with the detectable color change being visible via the indicator so as to provide a visual indication of the tobacco smoke.

2. The tobacco smoke detector of claim 1, further comprises a housing adapted to contain the sensor and the indicator.

3. The tobacco smoke detector of claim 1, wherein the reagent comprises at least an enzyme or a protein or a chemical substance.

4. The tobacco smoke detector of claim 1, wherein the sensor is integrated with the indicator.

5. The tobacco smoke detector of claim 1, is a self-contained unit allowing for easy transport, installation, and replacement.

6. The tobacco smoke detector of claim 1, is tamper resistant.

7. The tobacco smoke detector of claim 1, wherein the biological or chemical component is capable of initiating a series of chemical reactions in the presence of tobacco smoke or compounds unique to tobacco smoke.

8. The tobacco smoke detector of claim 2, wherein the housing comprises a transparent window for the indicator.

9. The tobacco smoke detector of claim 2, wherein the housing comprises a self-adhesive layer.
